(19) United States
(12) Patent Application Publication
(10) Pub. No.: US 2013/0338699 A1
(43) Pub. Date: Dec. 19, 2013

(54) VASCULAR OCCLUSION DEVICE

(71) Applicant: COOK MEDICAL TECHNOLOGIES LLC, Bloomington, IN (US)

(72) Inventors: Jacob Lund Clausen, Herlev (DK); Per Hendriksen, Herlufmagle (DK); Jeppe Boeckhaus Johnsen, Froerup (DK); Nicholas Gulmann Lundsteen, Hvalsoe (DK); Bent Oehlenschlaeger, Skensved (DK)

(73) Assignee: Cook Medical Technologies LLC, Bloomington, IN (US)

(21) Appl. No.: 13/803,953

(22) Filed: Mar. 14, 2013

(30) Foreign Application Priority Data
Jun. 14, 2012 (GB) GB 1210562.3

(51) Int. Cl. A61B 17/12

(52) U.S. Cl. CPC A61B 17/12109
USPC 606/198

(57) ABSTRACT

A vascular occluder includes a frame (10) having a generally cylindrical body portion (12), first and second end conical portions (14, 16) and first and second extremities (18, 20). Located within the fame (10) is a fibrous barrier (28) which provides rapid occlusion. The extremities (18, 20) are coupled by a coil spring (22) which acts to pull the extremities (18, 22) towards one another and thereby to cause longitudinal contraction an consequential radial expansion of the frame (10). The coil spring (22) ensures rapid and reliable deployment of the occluder in a patient's vessel. The occluder also can be compressed to a narrow diameter for deployment by means of narrow diameter sheaths.

This application claims the benefit of the filing date of United Kingdom (GB) patent application number 1210562.3, filed Jun. 14, 2012, which is hereby incorporated by reference herein.

### TECHNICAL FIELD

The present invention relates to a vascular occlusion device and to an introducer assembly including such a device.

### BACKGROUND ART

In a number of medical conditions and surgical procedures it is required or advisable to occlude a patient's vessel, be it temporarily or long term. For this purpose, vascular occlusion devices are known which are able to be deployed endoluminally in a patient. A variety of occlusion devices is known, including devices which provide substantially instantaneous occlusion, by means of an impermeable barrier, to devices which achieve occlusion over time, particularly by promoting blood clotting at the device, which blood clotting provides the desired occlusion of the vessel.

Endoluminal delivery of medical devices requires the meeting of a number of criteria for optimum performance. For instance, effective endoluminal delivery should be by means of a flexible introducer assembly able to curve and bend with the patient's vasculature from the percutaneous entry point to the location at which the device is to be deployed. A flexible introducer assembly generally also requires the device itself to be flexible when radially compressed into the introducer assembly. The device must also be able to be compressed radially into small diameter so as to be able to pass easily into and through a patient's vessels, particularly in the cases where these narrow. It is also important that the occluder be deployed reliably so as to avoid any loss of occlusion function which may result in an abortive procedure. In the case of temporary occluders, it is important to be able to remove the occluder readily at the end of its period of use, most preferably by means of a further endoluminal procedure so as to minimise trauma to the patient.

The structure of many occluder devices results in them being relatively big when compressed for delivery, caused in part by the number of elements forming the device, the need for the occluder to be particularly strong to be able to withstand and counter fluid forces within the vessel and so on. These features can lead to restrictions in compressibility as well as loss of flexibility of the device, particularly when compressed for delivery.

Examples of prior art devices can be found, for example, in U.S. Pat. No. 6,949,116, GB-1,333,096, US-2008/0065148, US-2010/0106178, U.S. Pat. No. 7,278,430, U.S. Pat. No. 6,551,344 and U.S. Pat. No. 7,572,272.

### DISCLOSURE OF THE INVENTION

The present invention seeks to provide improved vascular occlusion.

According to an aspect of the present invention, there is provided a vascular occlusion device including first and second end members movable relative to one another, a configurable frame coupled between the first and second end members, which frame is configurable between an elongated radially contracted configuration and a radially expanded longitudinally contracted configuration in dependence upon the positions of the first and second end members relative to one another; a fibrous barrier member carried by the frame; wherein the frame, when in the radially expanded configuration, radially expands the barrier so as to provide an occlusion function.

The frame has a configurable structure able to provide occlusion by a simple mechanical movement of the end members, in which occlusion occurs by means of the fibrous barrier. The structure moreover enables the use of a frame made of fine material which is able to be compressed radially to a relatively small diameter, less than is possible with prior art structures. The fibrous barrier member provides similar advantages.

Advantageously, the device includes a configuration mechanism coupled to the first and second end members and operable to cause the first and second end members to move towards one another so as to change the configuration of the frame.

In one embodiment, the configuration mechanism includes a biasing element operable to impart a biasing force to the first and second end members so as to move the end members towards one another. The biasing member may be a spring coupled between the first and second end members, the spring preferably being a coil spring.

The biasing member ensures deployment of the device, that is its radial expansion, allowing the use of a frame which is not necessarily able to impart an adequate deployment force per se. This also adds to the ability to minimise the structure and thus volume of the frame and to minimise the radially contracted footprint of the device for deployment. It can also lead to a highly configurable frame able to adapt to the shape and contour of the vessel in which it is deployed.

In the preferred embodiment, the frame is generally cylindrical in at least one configuration thereof. A cylindrical occlusion device can ensure reliable positioning in a vessel.

The first and second end members may be a part of the frame and in another embodiment may be tubular elements coupled to the frame.

The device may include a carrier element, the first and second end members being disposed on the carrier element and at least one of which is movable on the carrier element. The carrier element could be a cannula and may have the optional function of acting as a guide wire cannula. The carrier element may be a part of one of the occlusion device and an introducer assembly.

Thus, advantageously, the first and second end members allow for over the wire deployment, in an embodiment the carrier element being a cannula disposed between the first and second end members and through which a guide wire may pass and in another embodiment the carrier element being a guide wire catheter passing through the first and second end members.

In the preferred embodiment, the frame is formed of a wire braid.

The barrier member may formed from a plurality of intertwined fibres, and in other embodiments may be formed from woven or knitted fibres.

In an embodiment, the fibrous member is woven, sutured or knitted to or otherwise intertwined with the frame.

The barrier may made of hydrophobic material, for instance a polymer material.

Preferably, the frame is made from a biocompatible metal or polymer, for example a spring or shape memory material, such as nickel titanium alloy, typically Nitinol; or an alloy made from cobalt, chromium, nickel, molybdenum and/or iron such as Elgiloy, Conichrome and Phynox.

Advantageously, the frame and the barrier may be co-braided to one another.

Another embodiment includes an actuating device provided with a push element and a pull element operable to push and pull the end members towards one another. The actuating element may be a part of the introducer assembly used for deploying the occlusion device.

### BRIEF DESCRIPTION OF THE DRAWINGS

Embodiments of the present invention are described below, by way of example only, with reference to the accompanying drawings, in which:

FIG. 1 is a side elevational view in partial cross-section of an example of frame structure for an embodiment of occlusion device as taught herein;

FIG. 2 is a side elevational view of the frame structure of FIG. 1;

FIG. 3 side elevational view of a preferred embodiment of vascular occlusion device in a longitudinally contracted configuration;

FIG. 4 is a side elevational view of the device of FIG. 3 in a longitudinally extended configuration;

FIG. 5 shows a first embodiment of a part of an introducer assembly for deploying an occluder of the type taught herein;

FIG. 7 shows another embodiment of a part of an introducer assembly for deploying an occluder of the type taught herein;

FIG. 7 is a side elevational view of another embodiment of occlusion device;

FIG. 8 is a side elevational view of the embodiment of occlusion device of FIG. 7 mounted on a guide wire catheter; and

FIG. 9 is a schematic view of a part of an embodiment of co-braided frame and barrier structure.

### DESCRIPTION OF THE PREFERRED EMBODIMENTS

It is to be understood that the drawings are schematic only and are not to scale. They are of a form which is intended to facilitate the understanding of the teachings herein.

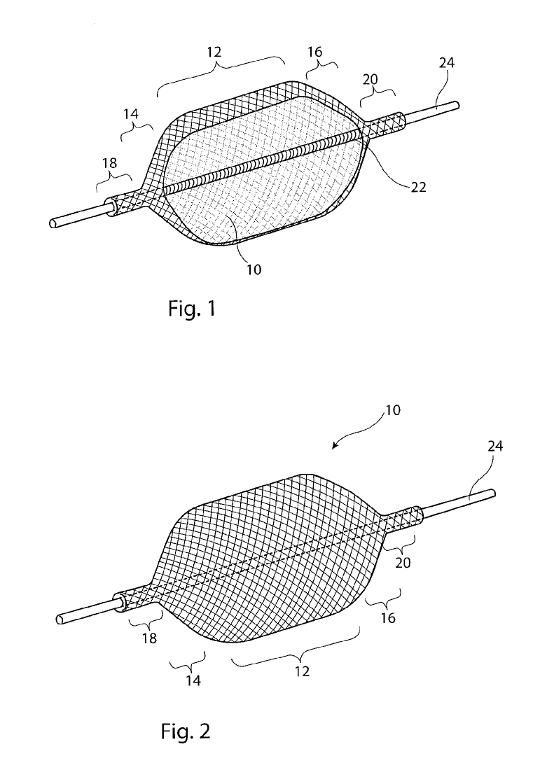

Referring to FIGS. 1 and 2, there is shown an embodiment of frame for an occluder assembly as taught herein. The frame 10 has a central generally cylindrical body portion 12 bounded by first and second tapered portions 14, 16 which end in frame extremities 18, 20. The frame 10 is formed from a wire braid, as are the tapered portions 14, 16. The extremities 18, 20 may equally be formed of braided wire, as extensions to the parts 12-16, but in other embodiments are tubular elements fixed to the braided wire, for instance by welding, soldering, bonding or any other suitable method.

As can be seen in particular in the cross-sectional view of FIG. 1, extending inside the frame 10 between the first and second extremities 18, 20, is a sprung element 22, in the example shown a coil spring. The sprung element 22 is fixed to the two extremities 18, 20.

FIGS. 1 and 2 also show the provision of a carrier element 24, being in this example a cannula or catheter. In the preferred embodiment, the carrier element 24 is a guide wire catheter of an introducer assembly used to deploy the occlusion device, whereas in other embodiments the carrier element 24 is a component separable from the introducer assembly used to deploy the occluder. At least one of the extremities 18, 20 is slidably disposed on the carrier element 24.

The frame 10 is preferably made from a biocompatible metal or polymer. It may be made from a spring or shape memory material. Preferred materials for the frame 10 include: a nickel titanium alloy such as Nitinol; or an alloy made from cobalt, chromium, nickel, molybdenum and/or iron such as Elgiloy, Conichrome and Phynox.

The sprung element may be made of a spring material such as spring steel or a shape memory material such as Nitinol.

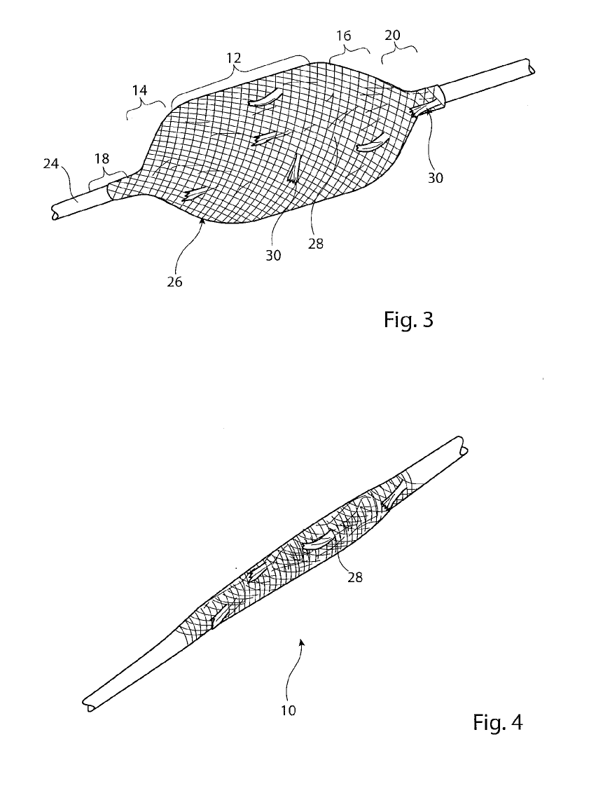

Referring now to FIG. 3, there is shown a side elevational view of an embodiment of occluder device 26, which includes a frame 10 having equivalent features to the frame 10 of the example of FIGS. 1 and 2, that is with a body portion 12, first and second tapering portions 14 and 16 and first and second extremities 18 and 20. Disposed within the frame 10 of the occluder device 26 is a fibrous barrier element 28. In this embodiment, the barrier element 28 is a mass of intertwined fine strands of a fibrous material, such as a polymer. The material is preferably a hydrophilic material. The mass of fine strands is such as to tend naturally to expand outwardly, in practice substantially to fill radially the space within the frame 10. On the other hand, the fibrous material of the barrier element 28 is also able to be compressed radially, as is explained below.

In the example shown in FIG. 3, the fibrous barrier element 28 is held substantially entirely within the frame 10. However, it is preferred that the barrier element 28 is in some way attached to the frame 10, particularly to the body portion 12 thereof. This may be by simple intertwining of the fibres of the barrier element 28 to the frame 10, by weaving, knitting or suturing the fibres to the frame 10 or by co-braiding with the wire forming the frame 10. FIG. 3 shows some of the fibres 30 extending out of the frame 10 as a result of such intertwining. It is to be understood that intertwining could be simply by pulling some of the fibres of the fibrous material through the spaces between the mesh of the braiding. Coupling the fibrous material to the frame 10 ensures that the fibrous material 10 will always radially fill the space within the frame 10.

As can be seen in particular in FIG. 3, the fibrous material of the barrier element 28 may extend the entire length of the frame 10, including the extremities 18 and 20, although this is not essential. In one embodiment, the fibrous material may be attached to the extremities 18 and 20.

Referring now to FIG. 4, the occluder device 26 of FIG. 3 can be seen in a longitudinally extended and radially contracted configuration, in which the frame 10 is disposed tightly against the carrier element 24, in what could be described a radially compressed configuration for deployment. The fibrous barrier 28 is also compressed radially inwardly as a result of the compression of the frame 26. It will be understood that this radial compression is achieved by elongation of the device 26 but in particular by pulling the two extremities 18, 20 away from one another. The fibrous barrier will be compressed by the frame and in embodiments where the barrier element is fixed to the extremities of the frame will also be pulled when the extremities move apart, causing the fibrous material to stretch longitudinally and compress radially.

In the case where one of the extremities 18, 20 is fixed to the carrier 24, narrowing of the frame can be achieved simply by pulling the other extremity in a device extending direction. Where both extremities 18, 20 are movable on the cannula 24, this extension can be achieved by pulling both extremities away from one another.

It will be appreciated in particular from FIG. 4 that the occluder can have a very small diameter when compressed, enabling it to be deployed by means of a narrow diameter introducer assembly. Practical embodiments have already been constructed using a 4 French sheath (that is of a diameter of 1.3 mm). Moreover, the occluder 26 remains flexible when in a radially compressed configuration, which makes it suitable for deployment through tortuous vasculatures.

Of course, in the configuration in FIG. 4, the coil spring 22 held within the frame 10 is extended when the extremities are pulled apart, thereby to generate a contracting force. When the coupling or couplings pulling the extremities 18, 20 apart is or are released, the coil spring 22 will pull the extremities 18, 20 together again, thereby to cause the frame 10 to contract longitudinally and expand radially, thereby to attain again the configuration shown in FIG. 3. In so doing, the fibrous barrier 28 also expands with the frame 10 so as to provide an occlusion function.

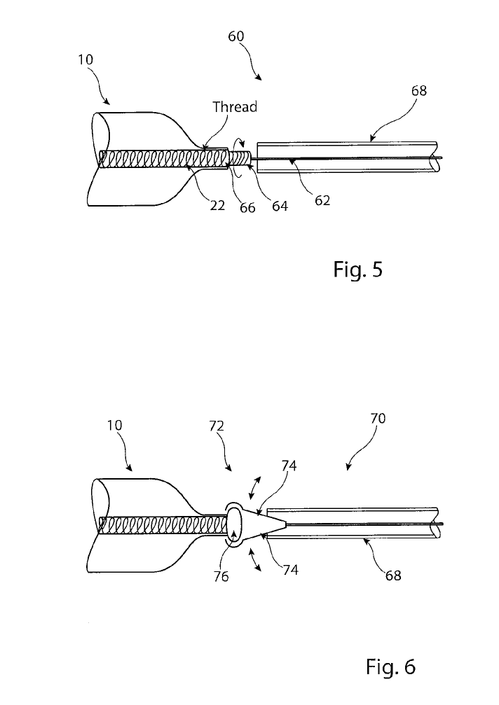

Referring now to FIGS. 5 and 6, there are shown two examples of the principal components of deployment assemblies for deploying an occlude of the type disclosed herein. FIG. 5 shows the part of the introducer assembly 60 which couples to the occludes 10. The assembly 60 includes a pusher pin 62 with thread 64 that connects to a threaded female terminal 66 of the occluder 10, the latter in one embodiment being a stabilised part of the coil spring 22. When the device 10 has been positioned at the intended site in the patient's vasculature, the occluder 10 is pushed out of the introducer sheath 68 and the pushing pin 62 is disconnected from the occluder device 10 by turning the pin/thread 64 out of the occluder device. If the location is not as desired, the occluder device 10 can be retracted for repositioning by being threaded again onto the pusher pin 62.

In FIG. 6, the introducer assembly 70 includes a spring force operated mechanism 72 which when in its relaxed position, as shown, is opened. Gripper arms 74 of the mechanism 72 fit around a knob 76 on the occluder device 10. When the coupling the mechanism 72 is retracted into the sheath 68, the arms 74 close onto the knob 66 and thus grip the occluder device 10 thereto. The occluder device 10 can thus be repositioned and can also be withdrawn completely from the patient's vasculature. When release mechanism 72 has been fully opened, the occluder device 10 is disconnected from the release mechanism 72 and thus from the introducer assembly.

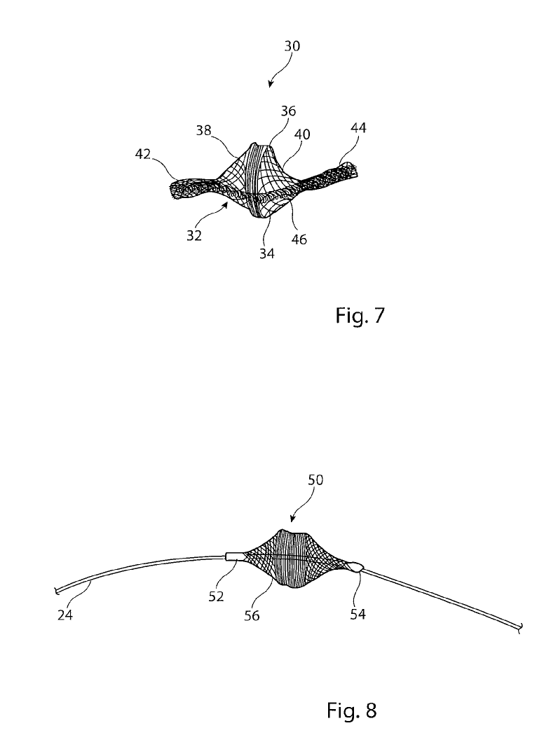

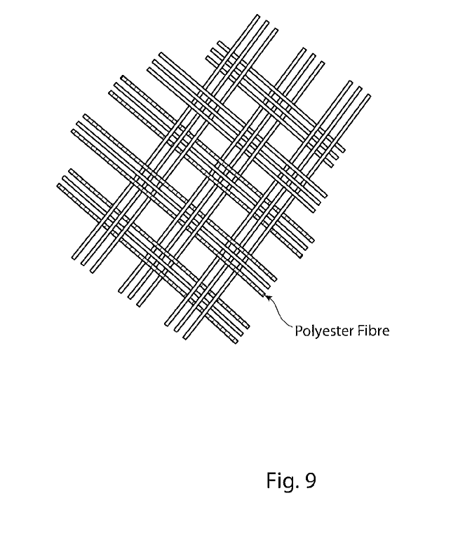

Further details of the deployment of the device 26 are described below following a description of the embodiment of FIGS. 7 and 8.

Referring now to FIG. 7, there is shown an embodiment of occluder device 30 which has similar characteristics to the occluder shown in FIGS. 1 to 4, that is having a frame 32 within which there is disposed fibrous barrier material 34. In this case, the frame 32 has a narrow body portion 36 and tapering portions 38, 40 coupling to the extremities 42, 44 respectively. As can be seen clearly in FIG. 7, the extremities 42, 44 are equally formed of the braided wire which forms the frame 32. The coil spring 46 can be seen inside the device 30.

FIG. 7 does not show any carrier cannula or catheter 24, for the reason that the device 30 is of a design which can be removed from the cannula or catheter 24, in which case the latter may usefully be a guide wire catheter of an introducer assembly.

The embodiment of FIG. 8 is virtually identical to the embodiment of FIG. 7, save for the fact that the extremities 52, 54 are tubular elements attached to the braiding forming the frame 56 of the device 50. The extremities 52, 54 can simply be short lengths of tubing which are welded, soldered or otherwise bonded to the frame 56. As with the embodiment of FIG. 7, the device 50 can fit onto a catheter 24, which in this embodiment is a guide wire catheter, and is able to slide on the catheter 24 so as to be completely removable therefrom once the occluder 50 has been deployed in a patient's vessel.

The deployment of an occluder device as taught herein can be effected by a relatively simple deployment procedure. As is conventional in the art of deployment of medical devices by means of an over-the-wire method, the preferred deployment procedure commences with disposing a guide wire in the intended vessel, typically by means of the Seldinger technique. Once the guide wire has been positioned, an introducer assembly with a preloaded occluder device is fed over-the-wire all the way to the intended deployment location. The introducer assembly will typically include a carrier catheter and an introducer sheath overlying the medical device during the deployment procedure.

Once the preloaded occluder has been positioned at the intended location, verified by means of contrast media and/or radiopaque markers in known manner, the introducer sheath is retracted to as to expose the occluder within the vessel. At this point, the occluder can be allowed to expand radially outwardly, which can be achieved by release of the extremities 18, 20 as appropriate. In other embodiments this may occur simply by retraction of the sheath, the sheath acting to maintain the occluder in a radially compressed configuration in the introducer assembly, and thus extremities 18, 20 apart from one another by means of the radial compassion force imparted by the sheath itself.

If it is determined that the occluder has not deployed at the correct location, it can be retrieved by being pulled back into the introducer sheath for repositioning. This is possible as the result of the fact that the occluder retains a narrow diameter extremity proximal end, which can be grabbed by any suitable grabbing element and pulled back into the sheath. The occluder can thus be redeployed to the correct location. Once at the desired location, the occluder can be fully detached from the introducer assembly and the guide wire catheter 24 retracted also, thereby to leave the occluder as a separate component within the patient's vasculature.

It will be appreciated that an over-the-wire system of the type described herein and shown in the drawings, will leave narrow apertures within the tubular extremities 18, 20. In many instances those apertures will not adversely affect the performance of the occluder. However, it is envisaged that in some embodiments there may be provided small valves within one or both of the extremities 18, 20, for example in the form of single or multi leaflet flap-type valves able to close once the guide wire catheter or other cannula has been removed from within the occluder.

The provision of the fibrous material within the frame of the occluder ensures faster occlusion of the vessel as the fibrous material not only acts as a barrier to particulate material but also promotes thrombus formation within the fibrous material. Thus, the occluder can provide rapid and in most instances virtually instantaneous occlusion of a vessel.

The provision of coil spring 22, 46 within the occluder ensures rapid radial expansion of the frame and also enables the frame to be made of very thin wire, having the advantage of being able to minimise the diameter of the device when radially compressed into an introducer assembly and thereby the footprint of the introducer assembly itself. A very thin wire braiding, if used alone, will not guarantee satisfactory expansion of the device during deployment as the result of the weakness of the wire. The coil spring 22, or other mechanism for pushing the two extremities 18, 20 towards one another, counters any weakness of the frame to radial expansion. Of course, the use of fine wire to make the frame also increases the flexibility of the device, facilitating its deployment through tortuous vessels.

Referring now to FIG. 9, there is shown the structure of another embodiment for the frame for an occlusion device. It is to be understood that the structure shown in FIG. 9 can be used in place of the frame and fibrous material shown in the embodiments of FIGS. 1 to 8. It is not excluded, however, that additional fibrous material of the type shown in particular in FIGS. 3 and 4 could be provided within the occluder. In the embodiment of FIG. 9, the frame is made of a co-braiding of metal or metal alloy wire and polyester fibres. In one example, one wire of Nitinol would be co-braided with one strand of polyester fibre. Other embodiments have a plurality of Nitinol wires to a single polyester fibre element, while other embodiments have a plurality of polyester fibres with one metal or alloy wire. In practice, the structure in FIG. 9 can be achieved by setting up a braiding machine such that the bobbins of the braiding machine are supplied with wire or polyester fibre and weaving the various wires/fibres together. Of course, the polyester fibres could be multi-stranded, wherein the different strands will splay outwardly to enhance the barrier effect.

An alternative to adding polymer fibres to the braiding process provides sewing or stitching fibres into the pre-braided wire frame as a final process in the assembly of the occluder device. It would only be necessary to stitch or sew fibres to the conical portions of the occluder device in order to obtain sufficient occlusion as the body portion of the device generally runs parallel to the blood vessel and thus has no effective occlusion function. Fitting the fibres to the frame after manufacture of the frame can avoid problems of heat setting of the frame, in the case where the frame is made of a shape memory material requiring such heat setting. Of course in the instance of use of a material which does not require heat setting or a material which is simply a spring material, heat setting would not be part of the production process.

It is to be appreciated that the fibrous barrier can be made of any suitable material, not just polymer such as polyester, preferably being a hydrophobic material to promote quick occlusion of the vessel.

It is to be understood that the features of the different embodiments described can be combined with one another and that the claims are to be interpreted, even though initially set out in single dependent form, as being combinable as if in multiple dependent form.

1. A vascular occlusion device including first and second end members movable relative to one another, a configurable frame coupled between the first and second end members, which frame is configurable between an elongated radially contracted configuration and a radially expanded longitudinally contracted configuration; a fibrous barrier member carried by the frame; wherein the frame, when in the radially expanded configuration, radially expands the barrier so as to provide an occlusion function.

2. A vascular occlusion device according to claim 1, including a configuration mechanism coupled to the first and second end members and operable to cause the first and second end members to move towards one another so as to change the configuration of the frame.

3. A vascular occlusion device according to claim 2, wherein the configuration mechanism includes a biasing element operable to impart a biasing force to the first and second end members so as to move the end members towards one another.

4. A vascular occlusion device according to claim 3, wherein the biasing member is a spring coupled between the first and second end members.

5. A vascular occlusion device according to claim 4, wherein the spring is a coil spring.

6. A vascular occlusion device according to claim 1, wherein the frame is generally cylindrical in at least one configuration thereof.

7. A vascular occlusion device according to claim 1, wherein the first and second end members are a part of the frame.

8. A vascular occlusion device according to claim 1, wherein the first and second end members are tubular elements coupled to the frame.

9. A vascular occlusion device according to claim 1, including a carrier element, the first and second end members being disposed on the carrier element and at least one of which is movable on the carrier element.

10. A vascular occlusion device according to claim 9, wherein the carrier element is a part of one of the occlusion device and an introducer assembly.

11. A vascular occlusion device according to claim 1, wherein the frame is formed of a wire braid.

12. A vascular occlusion device according to claim 1, wherein the barrier member is located in or on the frame.

13. A vascular occlusion device according to claim 12, wherein the barrier member is attached to the frame.

14. A vascular occlusion device according to claim 1, wherein the barrier member is formed from a plurality of intertwined fibres.

15. A vascular occlusion device according to claim 1, wherein the barrier member is made from woven or knitted fibres.

16. A vascular occlusion device according to claim 1, wherein the fibrous member is woven, sutured or knitted to or otherwise intertwined with the frame.

17. A vascular occlusion device according to claim 1, wherein the barrier is made of hydrophobic material.

18. A vascular occlusion device according to claim 1, wherein the barrier member is made of polymer material.

19. A vascular occlusion device according to claim 1, wherein the frame is made from a biocompatible metal or polymer.

20. A vascular occlusion device according to claim 1, wherein the frame is made from a spring or shape memory material.

21. A vascular occlusion device according to claim 1, wherein the frame is made from one or more of: Nitinol, Elgiloy, Conichrome and Phynox.

22. A vascular occlusion device according to claim 1, wherein the frame and the barrier are co-braided to one another.

23. A vascular occlusion device according to claim 1, including an actuating device provided with a push element and a pull element operable to push and pull the end members towards one another.
